(19) United States
(12) Patent Application Publication
(10) Pub. No.: US 2012/0301900 A1
(43) Pub. Date: Nov. 29, 2012

(54) APPARATUS AND METHOD FOR DETECTING TUMOR CELLS

(75) Inventors: Ji Yoon KANG, Seoul (KR); Sung Woo Lee, Daegu (KR)

(73) Assignee: Korea Institute of Science and Technology, Seoul (KR)

(21) Appl. No.: 13/479,987

(22) Filed: May 24, 2012

(30) Foreign Application Priority Data
May 27, 2011 (KR) 10-2011-0050506

(51) Int. Cl. C12M 1/34; G01N 33/574

(52) U.S. Cl. 435/7.23; 435/287.2

(57) ABSTRACT

Provided are an apparatus for detecting tumor cells including a tumor cell detection chip and a method for detecting tumor cells. The apparatus and method for detecting tumor cells according to the present disclosure enable convenient detection of tumor cells in short time and thus allow for treatment prior to metastasis of the tumor cells as well as easy diagnosis and clinical management of cancer patients. In addition, the detected tumor cells may be cultured as they are for use in genetic analysis.

### CROSS-REFERENCE TO RELATED APPLICATIONS

This application claims priority to Korean Patent Application No. 10-2011-0050506, filed on May 27, 2011, and all the benefits accruing therefrom under 35 U.S.C. §119, the contents of which in its entirety are herein incorporated by reference.

### BACKGROUND

1. Field

The present disclosure relates to an apparatus and a method for detecting tumor cells.

2. Description of the Related Art

Metastasis is the spread of cancer from the site where tumors originate to another part of the body. Some of these tumor cells travel via the peripheral blood to sites anatomically distant from the primary tumor. The disseminated individual cells present in small numbers may not be detected by standard methods such as microscopic examinations on dyed cyto-histological slides. Since detection and characterization of tumor cells are a promising method for both diagnosis and clinical management of cancer patients as well as treatment prior to metastasis, a new method for detecting tumor cells is required.

Circulating tumor cells (CTCs) are cells that have escaped from a primary tumor. While circulating in the blood or lymphatic vessels after passing through the mesenchymal-epithelial transition (MET) process, which is a cell structure change enabling metastasis, they penetrate into the endothelial cells at abnormal vessel walls (where inflammation or damage has occurred). At this time, they pass through the epithelial-mesenchymal transition (EMT) process. The EMT process is a process whereby cells lose their epithelial phenotype and convert to mesenchymal phenotype with increased cell mobility. It is known to be related with the metastasis of malignant tumors. The CTCs are transformed to a new tumor after passing through the EMT process and reside in another tissue as cancer. Accordingly, a method for detecting and characterizing the CTCs is useful not only in treatment prior to metastasis but also in diagnosis and clinical management of cancer patients. However, since the CTCs exist in around 100 cells per 1 mL of blood, it is not easy to detect them.

### SUMMARY

The present disclosure is directed to providing an apparatus and a method for easily detecting tumor cells conveniently in short time.

In one general aspect, the present disclosure provides an apparatus for detecting tumor cells in a sample, including a tumor cell detection chip, wherein the tumor cell detection chip includes: a substrate having an antibody fixed on one side thereof; and a chamber accommodating the substrate.

In an exemplary embodiment of the present disclosure, the chamber may comprise a sample inlet and a sample outlet.

In an exemplary embodiment of the present disclosure, the substrate may be surface-treated with a substance for inhibiting non-specific reactions.

In an exemplary embodiment of the present disclosure, the substance for inhibiting non-specific reactions may be bovine serum albumin (BSA) or polyethylene glycol (PEG).

In an exemplary embodiment of the present disclosure, the apparatus for detecting tumor cells may further include a device for applying centrifugal force to the tumor cell detection chip.

In an exemplary embodiment of the present disclosure, the substrate having the antibody fixed may be located along a direction where the centrifugal force is applied.

In an exemplary embodiment of the present disclosure, the apparatus for detecting tumor cells may further include a gas injector injecting a gas into the chamber through the sample inlet so as to discharge unreacted cells through the sample outlet.

In an exemplary embodiment of the present disclosure, the chamber may have a volume of 1-8 mL.

In another general aspect, the present disclosure provides a method for detecting tumor cells in a sample, including adding a sample to an antibody binding specifically to the tumor cells and applying centrifugal force to react the tumor cells with the antibody.

In an exemplary embodiment of the present disclosure, the centrifugal force may be 0.6-10 G.

In an exemplary embodiment of the present disclosure, the centrifugal force may be applied for 1-10 minutes.

In an exemplary embodiment of the present disclosure, the volume of the sample may be 1-8 mL.

In an exemplary embodiment of the present disclosure, the tumor cells may be circulating tumor cells (CTCs).

In an exemplary embodiment of the present disclosure, the method for detecting tumor cells may further include, after the adding a sample to an antibody binding specifically to the tumor cells and applying centrifugal force, washing the chamber by applying a gas to the sample to discharge unreacted cells.

In an exemplary embodiment of the present disclosure, the flow rate of the gas may be 3-10 mL/hr.

In an exemplary embodiment of the present disclosure, the flow rate of the gas may be a flow rate at which the difference of the capture rate of the tumor cells and the capture rate of non-tumor cells is maximum when the number of cells in the sample is known, the capture rate being defined by the equation 1:

$$Capture rate ( \% ) = \frac{Number of cells in a chip after washing}{Number of cells in a sample} \times 100 \qquad [ Equation 1 ]$$

In an exemplary embodiment of the present disclosure, the gas may be air, nitrogen or an inert gas.

In an exemplary embodiment of the present disclosure, the method for detecting tumor cells may further include, after the washing, culturing the captured tumor cells as they are.

In an exemplary embodiment of the present disclosure, the culturing may be performed for at least 3 days, specifically for at least 5 days.

In an exemplary embodiment of the present disclosure, the detecting is performed by using the apparatus for detecting tumor cells disclosed herein.

### BRIEF DESCRIPTION OF THE DRAWINGS

The above and other objects, features and advantages of the present disclosure will become apparent from the following description of certain exemplary embodiments given in conjunction with the accompanying drawings, in which:

FIG. 1 shows a tumor cell detection chip 10;

FIG. 2 shows a rotating device for applying centrifugal force to the tumor cell detection chip 10;

FIG. 3 schematically illustrates a method for detecting tumor cells;

FIG. 4 shows immunofluorescent staining for confirming tumor cells [(a)-(c): NSCLC cells; (d)-(e): Jurkat cells];

FIG. 5 shows the effect of centrifugal force on cells;

FIG. 6 shows separation of cells at air interface in an apparatus for detecting tumor cells;

FIG. 7 shows capture count and capture rate of tumor cells;

FIG. 8 shows capture rate of tumor cells in whole blood;

FIG. 9 shows proliferation of captured cells after culturing; and

FIG. 10 shows a relationship between the detection rate of NSLCL cells and the size of a tumor cell detection chip.

### DETAILED DESCRIPTION OF MAIN ELEMENTS

10: tumor cell detection chip
20: jig for fixing tumor cell detection chip
30: fixing means
40: rotating plate
50: supporting means
60: fixing means
102: sample inlet
104: sample outlet
106: substrate having antibody fixed thereon
108: chamber

### DETAILED DESCRIPTION

Hereinafter, the exemplary embodiments of the present disclosure will be described in detail with reference to the accompanying drawings.

The present disclosure provides an apparatus for detecting tumor cells in a sample, comprising a tumor cell detection chip 10, wherein the tumor cell detection chip 10 comprises: a substrate 106 having an antibody fixed on one side thereof; and a chamber 108 accommodating the substrate 106.

The term “antibody” described herein may be a monoclonal antibody or a polyclonal antibody. The antibody may be fixed on a solid substrate 106. As used herein, the term “substrate” refers to a mixing means having a non-biological, synthetic, planar and flat surface. It may have hybridization or enzyme recognition sites or other various recognition sites. The substrate may comprise, for example, a semiconductor, (organic) synthetic metal, synthetic semiconductor, insulator or dopant, metal, alloy, element, compound or mineral. It may be a synthesized, etched, lithographed, printed or microfabricated slide and may comprise a polymer, plastic, membrane, silicon, silicate, PMMA, PDMS, glass, metal, ceramic, wood, paper, hardboard, cotton, wool, cloth, or woven or nonwoven textile or fabric, but is not limited thereto.

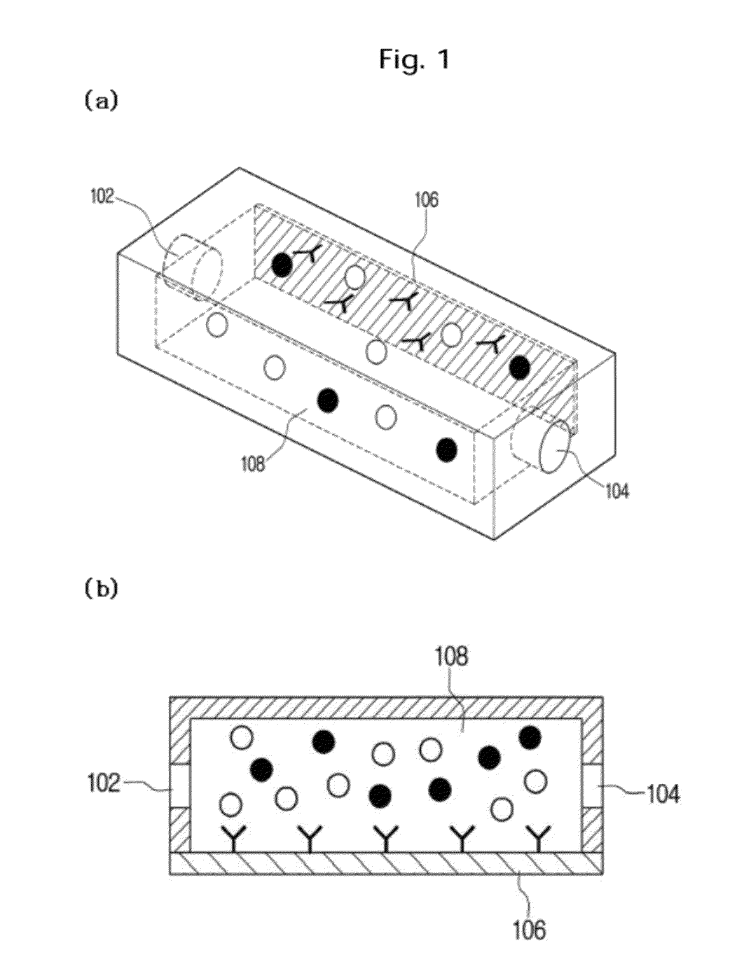

In the tumor cell detection chip 10, the chamber 108 accommodates the substrate 106 having the antibody fixed thereon. The accommodation herein may mean that the substrate 106 forms one side of the chamber 108 such that the side of the substrate 106 with the antibody fixed faces toward the inside of the chamber 108 (FIG. 1(a)). Alternatively, the chamber 108 may surround the substrate 106 on which the antibody is fixed (FIG. 1(b)).

The chamber 108 may comprise a sample inlet 102 and a sample outlet 104. For example, the sample inlet 102 and the sample outlet 104 may be formed at opposite ends of a line in the chamber 108, as shown in FIG. 1(a), but without being limited thereto.

The chamber 108 may have a volume of 1-8 mL. The volume of a sample to be detected may also be 1-8 mL. This numerical value means the volume of the sample from which tumor cells may be detected at once for a short time.

For accurate detection of circulating tumor cells (CTCs), which are present in trace amounts, a sample with a volume of mL scale is required. A microfluidic chip requires a long time for all the cells to react in a confined space. Furthermore, in a structure using reaction with antibodies, a slower flow rate is required since the cells become distant from the antibody-coated surface due to shear force. However, since the tumor cell detection chip of the present disclosure is irrelevant to the microfluidic environment, detection time may be decreased by increasing the flow rate. The tumor cell detection chip of the present disclosure allows for injection of a sample with a volume of 1 mL or larger at once and fast reaction of the whole sample on the antibody-coated surface using centrifugal force. In addition, it requires no difficult and complicated manufacturing as in microstructures and may be manufactured easily since no additives such as magnetic beads are necessary.

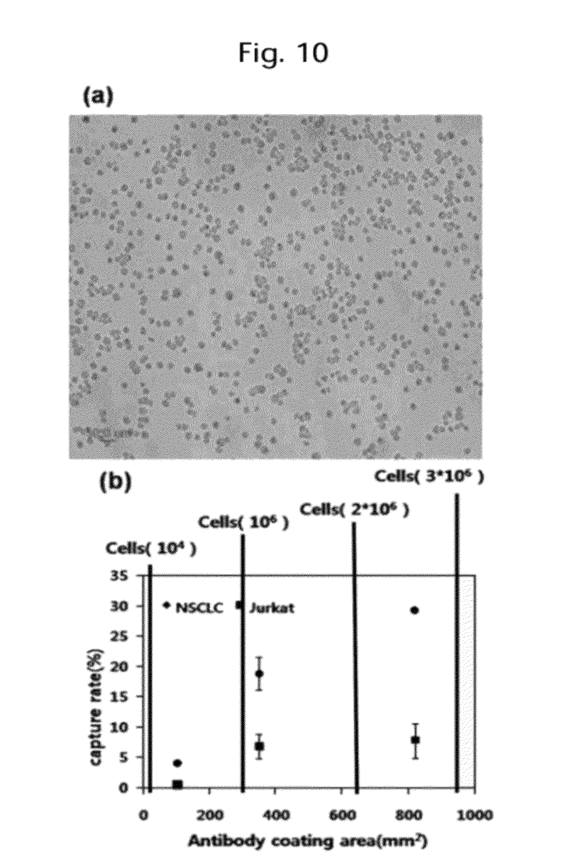

The area of the substrate 106 may be controlled according to the number of the cells included in the sample. It is because, since all the sample is injected to the chip at once, the cells adhere to the antibody at once rather than reacting sequentially with the antibody. As seen from FIG. 10(b), it is necessary to increase the area of the substrate or decrease the number of the cells in the sample in order to increase the detection rate at the detection chip. Further, the cells should be distributed well in the sample. If the cells are aggregated, the detection efficiency decreases since they will adhere to the antibody-coated surface at the same time. The aggregated cells may not react with the antibody but remain adhering to one another.

The substrate 106 may be surface-treated with a biochemical substance for inhibiting non-specific reactions. The substance for inhibiting non-specific reactions may be bovine serum albumin (BSA) or polyethylene glycol (PEG). The surface treatment may be performed by coating. The BSA or PEG may be used after being diluted. The BSA is used to prevent non-specific binding such as undesired antigen-antibody reaction on the substrate 106 and also as a complement of protein (enzyme) concentration or a nutrient during the culturing of the captured tumor cells.

The sample may be a blood sample possibly including the tumor cells.

The tumor cells may be CTCs, but are not limited thereto.

The substrate 106 having the antibody fixed may be located along a direction where centrifugal force is applied.

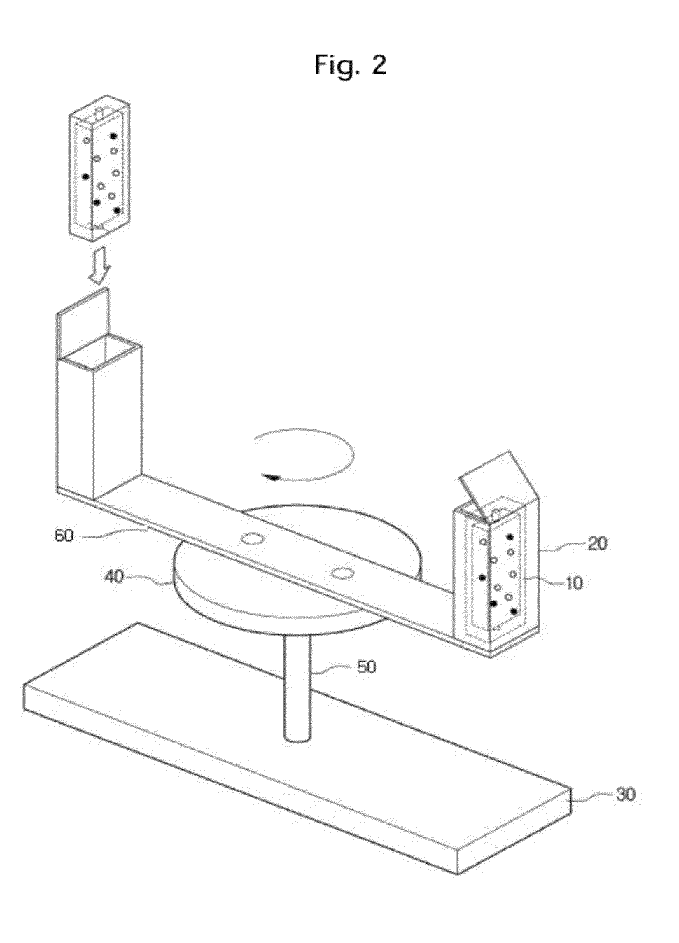

A device for applying centrifugal force to the tumor cell detection chip 10 is shown in FIG. 2. Referring to FIG. 2, the device may comprise a supporting means 50 vertically connecting a fixing means 30 with a rotating plate 40. A fixing means 60 may be fixed horizontally on the rotating plate 40 and chamber-shaped jigs 20 vertically fixing the tumor cell detection chip 10 may be provided at both ends of the fixing means 60.

The apparatus for detecting tumor cells may further comprise a gas injector injecting a gas into the chamber 108 through the sample inlet 102 so as to discharge unreacted cells through the sample outlet 104. The gas injector may be, for example, a syringe pump.

The unreacted cells refer to the cells remaining without reacting with the antibody and may be non-tumor cells. The non-tumor cells may be, for example, red blood cells, white blood cells, lymphocytes, or the like.

The gas may be air, nitrogen or an inert gas.

The present disclosure further provides a method for detecting tumor cells in a sample, comprising a step of adding a sample including tumor cells to an antibody binding specifically to the tumor cells and applying centrifugal force to react the tumor cells with the antibody.

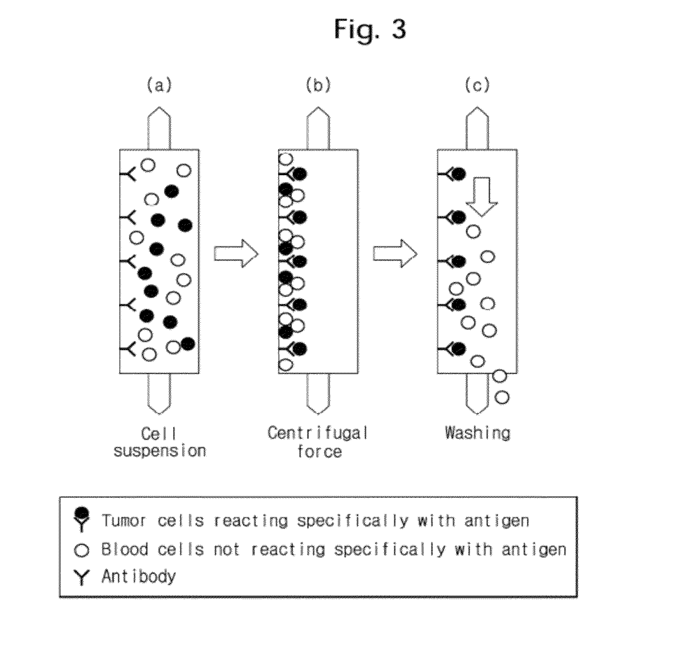

For detection of CTCs, a specific antibody acting on the cell membrane of tumor cells may be used. And, in order to enhance adhesion of the antibody to the cell membrane of the tumor cells, high pressure is generated by applying centrifugal force such that the tumor cells are attached well to the antibody. Referring to FIG. 3(b), after application of centrifugal force, all the cells in the sample are shifted toward the direction of the centrifugal force application. Hence, the tumor cells bind better to the antibody located along a direction where the centrifugal force is applied. The method of the present disclosure improves adhesion between the antibody and the cell membrane of tumor cells by approximately 10 times as compared to the existing method for enhancing the adhesion between the antibody and the cell membrane of the tumor cells in a microstructure.

Since the CTCs are generally present in trace numbers, a large amount of sample is required for detection. In order to process the large amount of sample fast, the present disclosure employs a method of injecting the large-volume sample at once. The existing methods for detecting tumor cells still have many problems. The most commonly used methods based on antigen-antibody reaction include: a method of using cell adhesivity and fluid shear force around cells based on microfluidics in a microstructure; a method of attaching cells on magnetized beads on which antibodies are attached and separating them using an electromagnet; and a method of additionally using special proteins for antigen-antibody reaction. However, these methods have some problems. First, the method using a microdevice or a microstructure requires long sample processing time (2 or more hours for 1 mL of sample) due to low flow rate. Further, the force for colliding the tumor cells with the antibody-coated microstructure is weak. In order to increase this force, the flow rate should be increased, which results in detachment of the tumor cells. The non-microfluidics-based method using magnetic beads requires magnetized beads and long incubating time is necessary for binding with antibodies. In addition, the magnetized beads affect the following procedures since they remain attached.

The existing methods for detecting tumor cell wherein antibodies are not used include a filtering method based on the size of tumor cells and a method of using a device having a special surface structure. Also, there is a method of using an aptamer for adhesion instead of antigen-antibody reaction. Finally, there is a method of separating tumor cells using the electrical properties of the cell membrane of tumor cells. However, these methods also have their problems. First, although the filtering method is based on the fact that tumor cells are generally larger than white blood cells in size, purity may be low since the tumor cells have varying size. That is to say, separation from other blood cells is not easy. The method using the surface structure requires a long detection time since microfluidics is used and the manufacturing of the device is complicated. The method using the aptamer is problematic in that the kind of available aptamers is few and the detection time is long since a long time is needed for reaction between the aptamer and the cell membrane of tumor cells. Finally, since the method using the electrical properties of the cell membrane of tumor cells is accompanied by deformation or damage of cells, the cells cannot be used in the following procedures.

The detection method of the present disclosure solves all the problems of low purity of the filtering method, long detection time or complicatedness of device manufacturing, and cell damage.

In the present disclosure, centrifugal force is used to enhance adhesion of the antibody to the cell membrane of tumor cells, and a gas is used in separation of blood cells to decrease detection time.

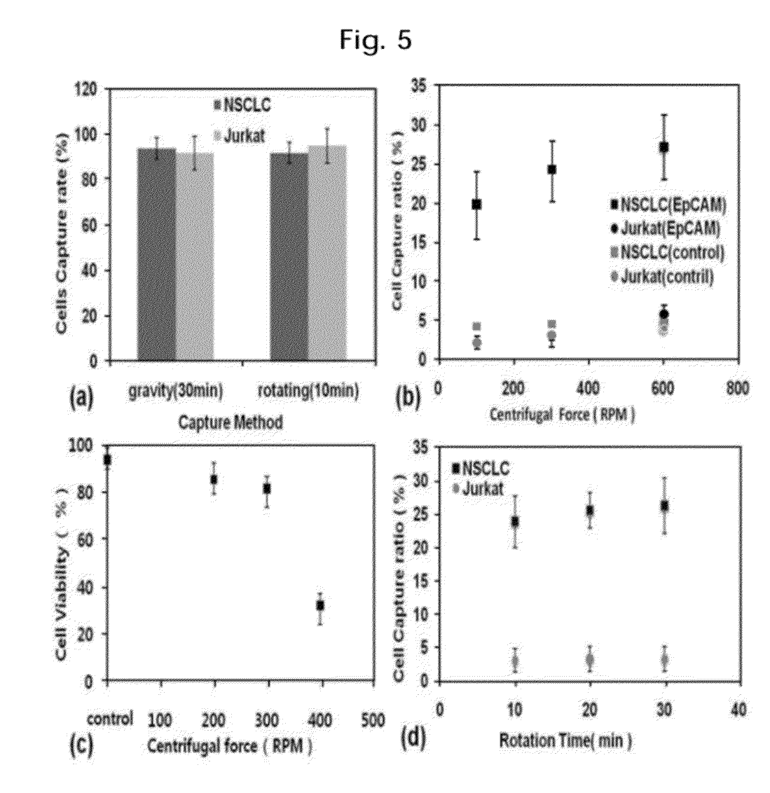

In the existing methods, a microstructure is disposed on the surface where cells flow and the surface is coated with antibodies in order to promote reaction between tumor cells and the antibodies. Such a microfluidic flow has the problem that the pressure that aids in the adhesion tends to be low since the sample flow rate is low. However, in the method of the present disclosure, since centrifugal force is applied in a direction where the substrate having the antibody fixed thereon is located, the pressure is increased remarkably and the cells may more easily move along the direction of the centrifugal force. That is to say, the physical pressure may aid in cell capture together with the antibodies. This can be confirmed from comparison with gravity as seen from FIG. 5(a). The CTCs actually exposed to the high flow rate in the blood vessel will receive flow pressure from the blood when they adhere to the vessel walls.

In the present disclosure, the centrifugal force may be 0.6-10 G. If the centrifugal force is smaller than 0.6 G, the cells may not move fast. And, if it exceeds 10 G, the cells may be damaged and, as a result, purity of the captured tumor cell may be unsatisfactory.

The centrifugal force may be applied for 1-10 minutes. If the centrifugal force is applied for less than 1 minute, the effect of applying the centrifugal force may be insignificant. And, even if it is applied for longer than 10 minutes, there is little difference in effect.

The method for detecting tumor cells may further comprise, after the step of reacting the tumor cells with the antibody, a washing step of discharging unreacted cells by applying a gas to the sample. Referring to FIG. 3(c), it can be seen that unreacted cells that did not react with the antibody are discharged by gas washing. The unreacted cells that did not react with the antibody may be non-tumor cells. The non-tumor cells may be, for example, red blood cells, white blood cells, lymphocytes, and so forth.

In the washing step of the method for detecting tumor cells, the gas may be injected through the sample inlet 102 when the apparatus for detecting tumor cells described above is used. The unreacted cells that did not react with the antibody may be discharged through the sample outlet 104.

The gas may be air, nitrogen or an inert gas.

The air washing method using interfacial tension and shear force caused by air flow is advantageous in that no damage is done to the cells. The reason why cells are detached is related not only with the shear force of fluid but also with the interfacial tension between two fluids (i.e. liquid sample and air). If the washing is performed using a liquid having similar properties as water, the effect of washing is insignificant since no interfacial tension is generated. When a gas, not liquid, is flown along the wall of a container filled with a liquid, interfacial tension is generated between the two fluids. Then, among the cells adhering to the wall, those not bound to the antibody are detached by the interfacial tension, as seen from FIG. 3(b) and (c).

At low flow rate, the shear force of fluid will have a greater effect since the cells are not detached easily. If the cells are exposed to air for a long period of time, the cells may die or be deformed, resulting in decreased adhesion between the cell membrane and the antibody. But, this problem does not occur when the exposure time is short.

If the centrifugal force is applied by rotating in the opposite direction, this force cannot generate shear force on the surface. Therefore, it is very difficult to detach cells by applying centrifugal force in the opposite direction and it is impossible to selectively remove the unreacted cells that did not react with the antibody.

In an exemplary embodiment of the present disclosure, the flow rate of the gas may be 3-10 mL/hr.

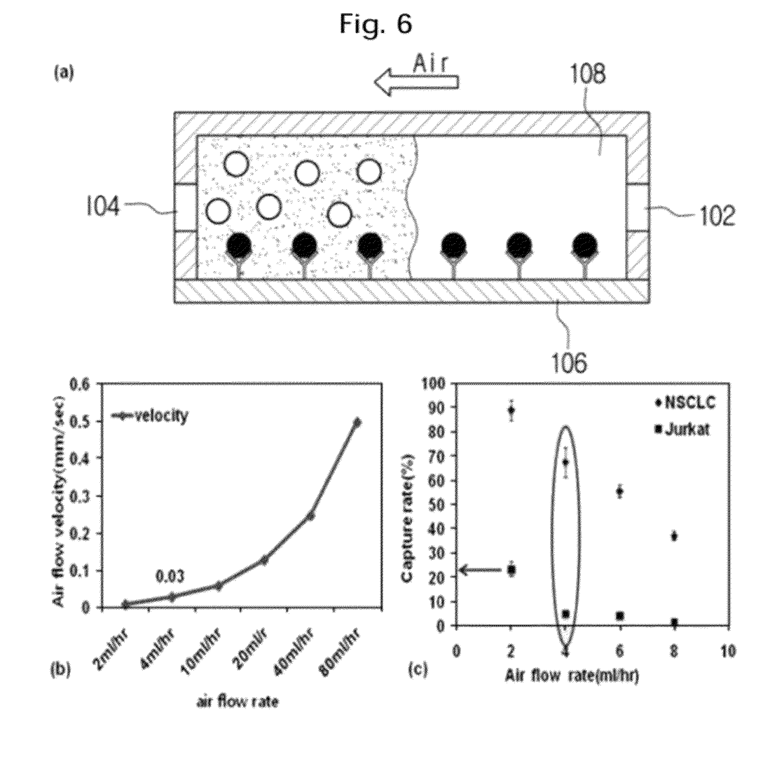

If the flow rate is lower than 3 mL/hr, non-tumor cells may not be removed well. And, if it exceeds 10 mL/hr, a large number of tumor cells may also be removed. Referring to FIG. 6(c), when the flow rate is 2 mL/hr, the capture rate of NSCLC cells (tumor cells) is about 90% and the capture rate of Jurkat cells (non-tumor cells) is over 20%. In 1 mL of actual blood sample, CTCs are present in less than 100 cells whereas non-tumor cells are present in more than million cells. Accordingly, if the capture rate of the non-tumor cells is 20%, detection of CTCs is practically difficult.

In an exemplary embodiment of the present disclosure, the flow rate of the gas may be a flow rate at which the difference of the capture rate of the tumor cells and the capture rate of non-tumor cells is maximum when the number of cells in the sample is known, the capture rate being defined by the equation 1:

$$Capture rate ( \% ) = \frac{Number of cells in chip after washing}{Number of cells in sample} \times 100 \qquad [ Equation 1 ]$$

The non-tumor cells mean the cells that did not react with the antibody. The non-tumor cells may be, for example, red blood cells, white blood cells, lymphocytes, and so forth.

In an exemplary embodiment of the present disclosure, the method for detecting tumor cells may further comprise, after the washing step, a step of culturing the captured tumor cells as they are. When the tumor cells are captured using the tumor cell detection chip 10, the tumor cells may be cultured as they are.

The tumor cells may be cultured for at least 3 days, specifically for at least 5 days. After the tumor cells are cultured for at least 3 days, they grow in number more than that of the tumor cells injected to the detection chip 10. And, if they are cultured for at least 5 days, normal blood cells are removed whenever the medium is replaced and only pure tumor cells remain on the substrate 106.

The captured tumor cells need to be cultured since they are too small in number to be subjected to genetic analysis such as PCR. The existing method of performing genetic analysis on the chip where the tumor cells are detected has a problem. For example, the characteristics of CTCs change when they metastasize to other organs via blood vessels. Thus, genetic analysis is required to identify their characteristics. And, since the CTCs have different characteristics when they emanate from the primary tumor, the analysis should be conducted for individual cells. However, since the tumor cells detected by the existing method are too few in number, it is impossible to perform PCR for individual cells. Thus, it is necessary to capture the tumor cells and proliferate the individual cells. According to the present disclosure, the tumor cells captured on the chip may be cultured directly without additional collection process. Even when the captured tumor cells are small in number, they proliferate very fast like normal tumor cells when they are cultured directly without collecting from the detection chip. Thus proliferated tumor cells may be used for various studies. Further, the analysis of the cells will be of great help in diagnosis and treatment of cancer patients.

The present disclosure will be described in further detail through examples and experiments. The following examples and experiments are for illustrative purposes only and those skilled in the art will appreciate that the scope of this disclosure is not limited by them.

### Test Example 1

### Tumor Cell Detection Chip Design and Surface Treatment

A tumor cell detection chip 10 used in the test was manufactured using a commonly used slide glass (3 mm×70 mm) as a substrate 106 and a chamber 108 was made of PDMS (FIG. 1). The inner size of the chamber was 1 cm×6 cm×0.5 cm (W×L×H) and the volume was 3 mL. The height of the chamber 108 is adjustable according to the volume of a sample. Holes of 2 mm in diameter were made on both ends of the chip to form a sample inlet 102 and a sample outlet 104. The chip 10 is characteristic in that the whole sample is reacted at once on the detection surface. Therefore, the surface area of the substrate 106 on which an antibody will be coated is restricted to an area of up to 1.9×10⁶ cells. With this area, all the cells included in a 1-mL sample solution can be accommodated enough excluding red blood cells. The surface of the substrate 106 on which the antibody will be coated was treated with S-adenosyl L-methionine (SAM).

First, the substrate 106 was treated with aminopropyltriethoxysilane (APTES) diluted to 1% in ethanol for 30 minutes. Then, the substrate 106 was exposed to a hot plate of 80° C. to evaporate ethanol for 1 hour for Le Chatelier reaction. Thus treated surface of the substrate 106 was immersed in glutaraldehyde diluted to 3% in distilled water (DW) for 1 hour, so that proteins could adhere on the surface. Then, the substrate 106 was washed with 1× phosphate-buffered saline (PBS) and DW. Then, the epithelial cell adhesion molecule (EpCAM), which is an antibody, diluted to 10 μg/mL in PDMS was coated on the surface of the substrate 106 for 30 minutes. Finally, the substrate 106 was immersed in 1% BSA for 1 hour and then washed with 1×PBS.

The EpCAM is an antibody reacting specifically with CTCs. Also, it reacts specifically with non-small-cell lung carcinoma (NSCLC) cells, which are human tumor cells. The NSCLC cells were used as CTCs.

### Test Example 2

### Cell Culturing and Sample Preparation

The human non-small-cell lung cancer (NSCLC) cell line NCI-H1650 was maintained and grown to confluence in RPMI-1640 medium containing 1.5 mM L-glutamine supplemented with 10% fetal bovine serum at 37° C. in 5% CO², with humidity according to the protocol provided by the manufacturer. The cell titre was determined by counting with a hemocytometer. The desired concentration of cells was then prepared by serial dilution of the original cell suspension in PBS. Cell viability was determined with the LIVE/DEAD viability assay. This assay is based on intracellular esterase activity of live cells and plasma membrane integrity of dead cells. Briefly, captured CTCs were incubated at room temperature for 30 min in a solution of 2 mM calcein FITC and 4 mM PI (Proliferation Index) prepared in PBS. At the end of the incubation period, the chip was washed with 1 ml of 1×PBS and visualized under the microscope. Labelled cells were spiked into whole blood.

Blood samples were drawn from healthy donors after obtaining informed consent without tumours, at Korea Institute of science and technology under an IRB-approved protocol. All specimens were collected into vacutainer tubes containing the anticoagulant EDTA and were processed within 24 h. Between sample collection and sample processing, whole blood specimens were stored at 4° C. on a rocking platform to prevent cell settling.

### Test Example 3

### Immunofluorescent Staining for Detection of CTCs

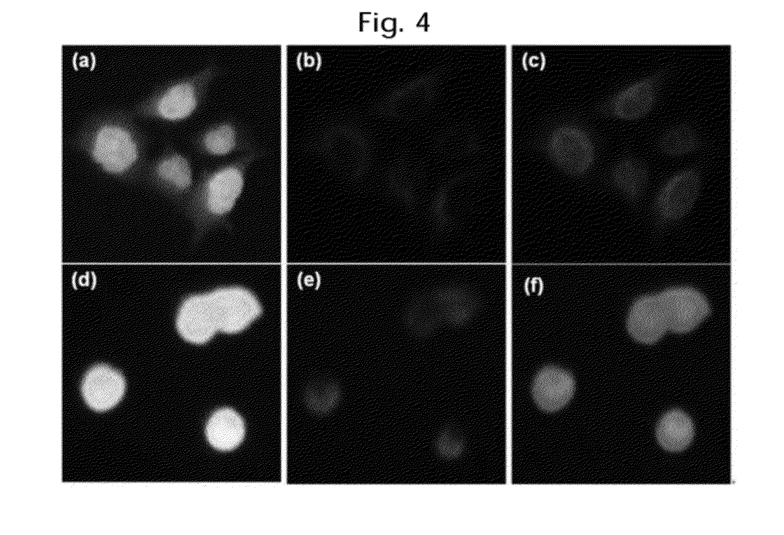

Captured cells were fixed by flowing 1 ml of 4% PFA in PBS, through the apparatus for 20 min. The chip was subsequently washed with a solution of 1 ml of 0.2% Triton X-100 in PBS for 30 min to induce cellular permeability and allow for intracellular staining. To identify any bound Jurkat cells or lymphocytes, 1 ml of anti-CD45FITC stock solution (50 ml of antibody stock solution in 1 ml of PBS) was passed through the chip for 2 hr, followed by a PBS wash to remove excess antibody (FIG. 4e). To identify epithelial cells, 1 ml of anti-cytokeratinPE stock solution (50 ml of antibody stock solution in 1 ml of PBS) was passed through the chip for 2 hr, followed by a PBS wash (FIG. 4b). Finally, to permit the identification of cellular nuclei 1 ml of DAPI solution (4 ml of DAPI reagent in 1 ml of deionized water) was passed through at the chip for 5 min, followed by a PBS wash (FIG. 4a, d). The chip was removed from the manifold, wiped dry near the fluid ports and stored in the dark at 4° C. until imaging. Biotinylated mouse anti-human anti-EpCAM was obtained from R&D Systems. Human non-small-cell lung cancer line NCI-H1755A, prostate cell line Jurkat clone E6-1 cell line were purchased from Korea Cell Line Bank, and RPMI-1640 growth medium was purchased from Invitrogen. Anti-cytokeratin PE (CAM 5.2, conjugated with phycoerythrin), CD45 FITC, the fluorescent nucleic acid dye nuclear dye 49,6-diamidino-2-phenylindole (DAPI) were purchased from BD Biosciences.

### Test Example 4

### Capturing of Cells Using Centrifugal Force and Air Interfacial Washing

1. Method

A device for applying centrifugal force to the detection chip was designed (FIG. 2). The device may accelerate up to 50-650 RPM and a chamber-shaped jig 20 for fixing the chip 10 was mounted on a rotating plate 40 (r=55 mm) FIG. 3 schematically illustrates the movement of cells toward the surface-treated substrate by the centrifugal force. (a) shows when a sample is injected to the tumor cell detection chip 10, and (b) shows the movement of the cells along the direction where the centrifugal force is applied. (c) shows the removal of the cells that did not react with the antibody by washing.

NSCLC cells (tumor cells) and Jurkat cells (non-tumor cells) of the same quantity in RPMI-1640 growth medium were injected into the tumor cell detection chip 10, and the detection chip 10 was placed in the jig 20 of the centrifugal force device. When the detection chip 10 is rotated, all the cells move along the direction where the centrifugal force is applied. When the cells move along the direction where the centrifugal force is applied and are located on the antibody-coated substrate 106, the NSCLC cells are adhered to the substrate as a result of antigen-antibody reaction (FIG. 3(b)), whereas the Jurkat cells that do not react with the antibody are discharged through the sample outlet of the detection chip by air washing (FIG. 3(c), FIG. 6(a)). The washing step has little effect on cell viability since cells are also exposed to air while old medium is replaced during the culturing of normal adherent cells. A syringe pump was used to inject air. The effect of the centrifugal force on the movement of cells was compared with that of gravity. Thus determined magnitude of centrifugal force is one under which all the cells are precipitated in the gravity experiment. Also, the effect of the magnitude of centrifugal force on cell viability was investigated. The experimental result is shown in FIG. 5.

2. Effect of Centrifugal Force on Cells

In order to identify whether all the cells in the sample move to the detection surface, a comparison was made with gravity. NSCLC cells and Jurkat cells of the same quantity were tested under centrifugal force and gravity environments. FIG. 5(a) shows cell capture rate under the centrifugal force and gravity environments. The experiment was performed using the tumor cell detection chip 10 and the number of cells was counted before washing. Under the gravity environment (1 G), it took 30 minutes for all the cells to move to the detection surface. However, under the centrifugal force environment of 300 RPM (5.5 G), the cells moved in 10 minutes (FIG. 5a). The cells move slightly faster under centrifugal force than they are precipitated under gravity. At 100 RPM (0.6 G), the cells cannot move fast since the centrifugal force is weak. When the rotating speed was increased to 600 RPM (22.1 G), the cells may be damaged. FIG. 5(b) shows cell capture rate under centrifugal force when antibody (EpCAM) was used. The cell detection rate relative to the extent of the centrifugal force was proportional to the rotating speed and the detection rate of NSCLC cells differed on the antibody-coated detection surface and on the non-coated surface (FIG. 5b). More NSCLC cells were detected on the antibody-coated surface. The detection rate of Jurkat cells was low regardless of the antibody. FIG. 5(c) shows cell viability before and after application of centrifugal force. FIG. 5(d) shows cell capture rate as a function of centrifugal force application time. When the centrifugal force exceeds 10 times of gravity (10 G), the condition of cells is aggravated rapidly. There was no significant difference when the centrifugal force application time was longer than 10 minutes (FIG. 5d).

3. Air Washing

Among the cells that moved to the antibody-coated detection surface of the substrate 106, the cells that did not react with the antibody were removed by air washing. FIG. 6(a) schematically illustrates removal of unreacted cells from the tumor cell detection chip by discharging using a gas. The air flows from the right side to the left side. The left space indicated by the blank is air and the right space indicated by small dots is filled with the sample. The cells shown as “•” are the NSCLC cells bound to the antibody (“Y”), and the cells shown as “∘” is the Jurkat cell removed by air. FIG. 6(b) shows air flow velocity inside the chip as a function of air flow rate. The velocity increases as the air flow rate increases, generating shear force on the detection surface. When the air flow rate was 4 mL/hr, the air flow velocity inside the chip was 0.03 m/s. If the air flow rate is too low, the Jurkat cells are not removed well. And, if it is too high, the NSCLC cells are also removed. FIG. 6(c) shows cell capture rate as a function of air flow rate. As the non-tumor cells are removed by air washing, only the tumor cells remain on the substrate 106.

### Test Example 5

### Detection of CTCs

1. Capture Rate, Detection Rate and Purity of CTCs

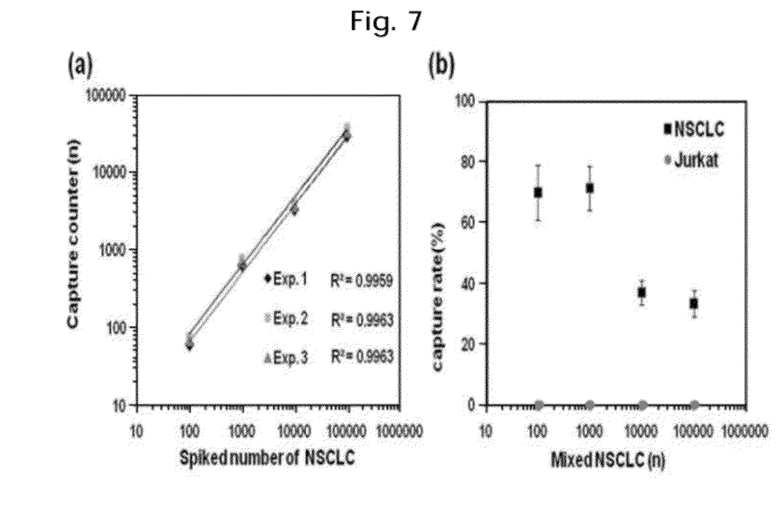

FIG. 7(a) shows the number of detected cells as a function of the number of NSCLC cells included in the sample. Since less than 100 CTCs are present in 1 mL of blood, 100-100,000 NSCLC cells were injected to the chip. And, 1,000,000 Jurkat cells were injected. The number of captured cells was relatively proportional to the number of injected cells. FIG. 7(b) compares capture rate of the NSCLC cells diluted in the sample with that of the Jurkat cells. The number of cells used was 10²-10⁵ for the NSCLC cells and 10⁶ for the Jurkat cells. But, the detection rate of the injected cells varied from 35% to 75% (FIG. 7(b)). It may be because the many NSCLC cells could not be distributed uniformly on the restricted detection surface. Even when the detection surface is infinitely large, the detection rate is not good if the cells remain attached. On the other hand, the cells are not captured well if the number of the cells is small. A detection rate of at least 70% was achieved at the level of cell number actually found in cancer patients. Referring to FIG. 7(b), detection rate of Jurkat cells was below 1% even when 1,000,000 cells were injected. The purity was 60%.

2. Detection Rate of CTCs in Whole Blood

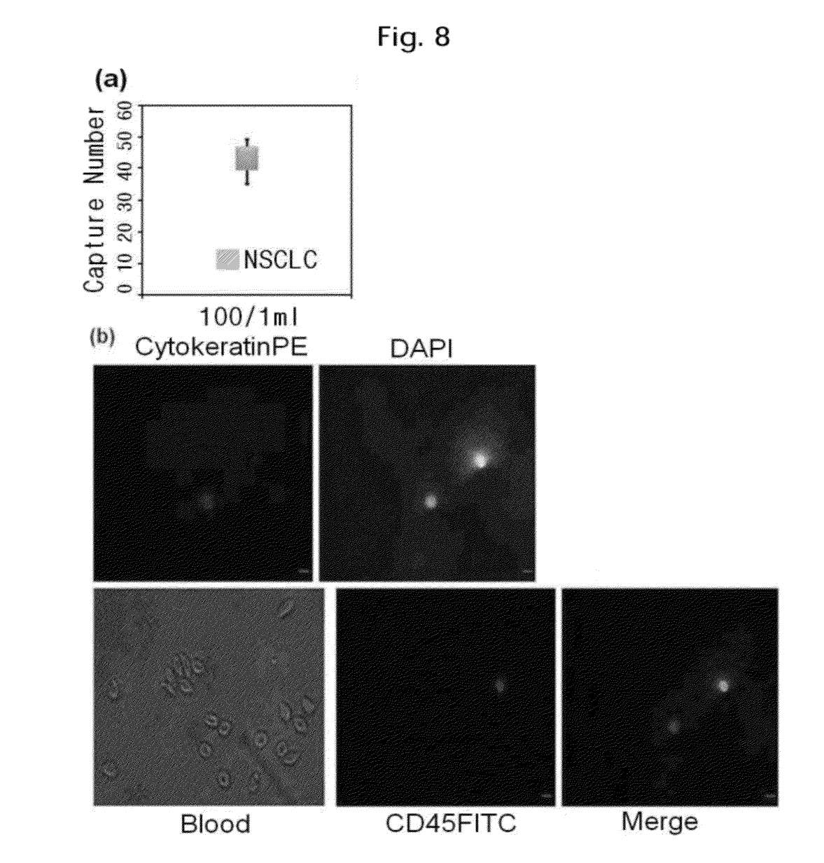

Finally, detection rate of tumor cells was tested after adding NSCLC tumor cells to the blood of healthy people. Whole blood was used without removing the red blood cells. One of the advantages of the tumor cell detection chip of the present disclosure is that a clinically obtained sample can be used directly without special treatment. The blood was mixed with 100 tumor cells and detection test was performed. FIG. 8(a) shows the number of captured NSCLC cells when 1 mL of whole blood was mixed with 100 NSLCL cells. The detection rate was about 45%. If the whole blood is diluted or the red blood cells are removed, a higher detection rate will be achieved. FIG. 8(b) shows fluorescence microscopic images of the detected cells. The NSCLC tumor cells are stained red by cytokeratin PE, the white blood cells are stained green by CD45 FITC, and the red blood cells remain unstained. And, all the cells excluding the red blood cell are stained blue by DAPI. If the whole blood is mixed with a diluting solution, a longer time will be required for washing. But, the time is shorter when compared with the existing detection method.

### Test Example 6

### Culturing of Detected CTCs

1. Method

It was investigated whether the tumor cells captured using the antibody and centrifugal force and separated by air washing can be cultured in the detection chip of the present disclosure as they are. Comparison was made with the existing tumor cell culturing method as control. The cells of the control group were grown in an ordinary culture vessel, whereas the tumor cells captured using the tumor cell detection chip of the present disclosure were grown on the antibody (EpCam) and 1% BSA coated on the slide glass. RPMI-1640 was used as growth medium. On days 1, 3 and 5, the culturing state of the cells was identified after staining and the number of the cells was counted. The medium was replaced every other day.

2. Evaluation of Proliferation and Purity of CTCs Cultured on Chip

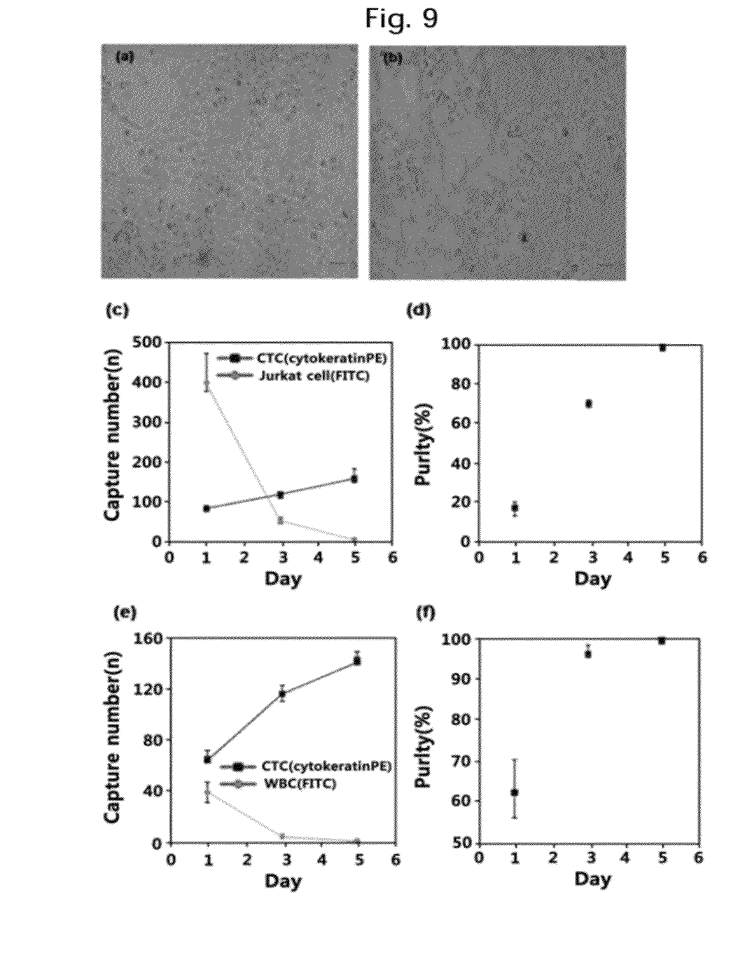

FIG. 9(a) shows a microscopic image of the tumor cells grown for 5 days in an ordinary culture vessel. FIG. 9(b) shows an image of the tumor cells grown for 5 days on the tumor cell detection chip. The captured tumor cells cultured on the detection chip proliferated well even after 5 days. The cells proliferated well on the antibody- and BSA-coated glass slide without being negatively affected. When compared with the tumor cells that grew adhering to the ordinary culture vessel, the proliferation rate was about 50% slower. FIG. 9(c) and FIG. 9(d) show the number of captured cells after the captured NSLCL and Jurkat cells were cultured for 1-5 days as well as the purity of the NSLCL cells. The number of the NSLCL and Jurkat cells used was 100 and 1,000,000, respectively. From day 3, the cells proliferated beyond the number of the tumor cells injected to the detection chip. However, the number of normal blood cells decreased rapidly since they fell off the glass slide when replacing the medium. Thus, after culturing for about 5 days, nearly all the normal blood cells fall off. As a result, only pure tumor cells remained on the chip. FIG. 9(e) and FIG. 9(f) show the result of capturing and culturing cells from a blood sample containing 100 NSLCL cells. In FIG. 9(a) and FIG. 9(b), the red bar is 50 μm in length.

FIG. 10(a) shows the area occupied by the detected cells on the detection surface of the chip. It is ideal in that the area occupied by the adhering cells is not larger than the area of the detection surface. FIG. 10(b) shows the upper limit of the number of cells that can adhere to detection surfaces of varying sizes as well as the actual detection rate of the captured cells. The number of the cells used is 1,000,000.

The apparatus and method for detecting tumor cells according to the present disclosure enable convenient detection of tumor cells in short time and thus allow for treatment prior to metastasis of the tumor cells as well as easy diagnosis and clinical management of cancer patients. In addition, the detected tumor cells may be cultured as they are for use in genetic analysis.

While the present disclosure has been described with respect to the specific embodiments, it will be apparent to those skilled in the art that various changes and modifications may be made without departing from the spirit and scope of the disclosure as defined in the following claims.

What is claimed is:

1. An apparatus for detecting tumor cells in a sample, comprising a tumor cell detection chip,
wherein the tumor cell detection chip comprises: a substrate having an antibody fixed on one side thereof; and a chamber accommodating the substrate.

2. The apparatus for detecting tumor cells according to claim 1, wherein the chamber comprises a sample inlet and a sample outlet.

3. The apparatus for detecting tumor cells according to claim 1, wherein the substrate is surface-treated with a substance for inhibiting non-specific reactions.

4. The apparatus for detecting tumor cells according to claim 3, wherein the substance for inhibiting non-specific reactions is bovine serum albumin (BSA) or polyethylene glycol (PEG).

5. The apparatus for detecting tumor cells according to claim 1, which further comprises a device for applying centrifugal force to the tumor cell detection chip.

6. The apparatus for detecting tumor cells according to claim 5, wherein the substrate having the antibody fixed is located along a direction where the centrifugal force is applied.

7. The apparatus for detecting tumor cells according to claim 2, which further comprises a gas injector injecting a gas into the chamber through the sample inlet so as to discharge unreacted cells through the sample outlet.

8. The apparatus for detecting tumor cells according to claim 1, wherein the chamber has a volume of 1-8 mL.

9. A method for detecting tumor cells in a sample, comprising adding a sample to an antibody binding specifically to the tumor cells and applying centrifugal force to react the tumor cells with the antibody.

10. The method for detecting tumor cells according to claim 9, wherein the centrifugal force is 0.6-10 G.

11. The method for detecting tumor cells according to claim 9, wherein the centrifugal force is applied for 1-10 minutes.

12. The method for detecting tumor cells according to claim 9, wherein the volume of the sample is 1-8 mL.

13. The method for detecting tumor cells according to claim 9, wherein the tumor cells are circulating tumor cells (CTCs).

14. The method for detecting tumor cells according to claim 9, which further comprises, after the adding a sample to an antibody binding specifically to the tumor cells and applying centrifugal force, washing the chamber by applying a gas to the sample to discharge unreacted cells.

15. The method for detecting tumor cells according to claim 14, wherein the flow rate of the gas is 3-10 mL/hr.

16. The method for detecting tumor cells according to claim 14, wherein the flow rate of the gas is a flow rate at which the difference of the capture rate of the tumor cells and the capture rate of non-tumor cells is maximum, the capture rate being defined by the equation 1:
$$Capture rate ( \% ) = \frac{Number of cells in a chip after washing}{Number of cells in a sample} \times 100 \qquad [ Equation 1 ]$$

17. The method for detecting tumor cells according to claim 14, wherein the gas is air, nitrogen or an inert gas.

18. The method for detecting tumor cells according to claim 14, which further comprises, after the washing, culturing the captured tumor cells as they are.

19. The method for detecting tumor cells according to claim 18, wherein the culturing is performed for at least 3 days.

20. The method for detecting tumor cells according to claim 18, wherein the culturing is performed for at least 5 days.

21. The method for detecting tumor cells, wherein the detecting is performed by using the apparatus for detecting tumor cells according to claim 1.
